(19) United States
(12) Patent Application Publication
(10) Pub. No.: US 2022/0293228 A1
(43) Pub. Date: Sep. 15, 2022

(54) Identification of Allergens in Food Products

(71) Applicants: Glenn Loomis, Cincinnati, OH (US); Kowsilliya Ramnaresh, Cincinnati, OH (US); Michael G. DiDonato, Fishkill, NY (US)

(72) Inventors: Glenn Loomis, Cincinnati, OH (US); Kowsilliya Ramnaresh, Cincinnati, OH (US); Michael G. DiDonato, Fishkill, NY (US)

(21) Appl. No.: 17/196,282

(22) Filed: Mar. 9, 2021

(51) Int. Cl. G16H 10/60; G16H 40/67; G16H 50/20; G16H 50/70; G16H 70/60

(52) U.S. Cl. CPC G16H 10/60; G16H 40/67; G16H 50/20; G16H 50/70; G16H 70/60

(57) ABSTRACT

A system for determining the presence or absence of allergens in a product is described. A memory is provided having at least one region for storing computer executable program code. A processor is provided for executing the program code stored in the memory, wherein the program code comprises:
accepting entry by the user of one or more allergens,
accepting entry of a product identification for a product,
finding a product matching the product identification in a database of product information including product ingredients,
comparing the product ingredients of the product to the one or more allergens entered by the user, and
   notifying the user of presence or absence of any of the one or more allergens in the product.

### TECHNICAL FIELD

This disclosure relates to allergies, and more particularly, to an application that will assist consumers in identification of food items that may contain ingredients to which they are allergic.

### BACKGROUND

Allergies are the 6^{th} leading cause of chronic illness in the United States, with an annual cost of $25 billion each year for food allergies. According to Food Allergy Research and Education, a food allergy is a serious challenge in the United States as 32 million Americans have food allergies, including 5.6 million children under the age of 18. This includes 1 in 13 children, or roughly two in every classroom. About 40% of children with food allergies are allergic to more than one food. Due to food allergy reactions, about 200,000 people require emergency medical care each year. A food allergy occurs when the body has a specific and reproducible immune response to a certain food or foods. The reactions can be severe and life threatening, such as anaphylaxis. For food allergic individuals, it can be a terrifying experience to try any new food because of the uncertainty about whether it contains their allergen.

Reading food labels to identify the ingredient(s) is critical for consumers with a food allergy. This allows consumers to identify and avoid foods that contain their allergen, which helps to prevent allergic reactions. The Food Allergen Labeling and Consumer Protection Act (FALCPA), a law implemented in January 2006, required eight major allergens be listed on food labels in easily understood language:
Peanuts
Tree nuts
Milk
Eggs
Wheat
Soy
Fish
Shellfish

Reviewing the top allergies and food items which consumers must learn to avoid daily in their routines has shown there is a common/usual name along with an allergen name which people with food allergies need to recognize in order to select the proper food item. As some food allergies can be categorized, such as wheat, there are also specific allergens which are variations of wheat proteins which people can avoid while still enjoying other wheat-based products.

Peanuts

Peanuts are most commonly associated with anaphylaxis, a sudden and potentially deadly condition that requires immediate attention and treatment. Peanuts and peanut-containing products can be found in candies, cereals, as well as baked goods such as cookies, cakes, and pies. Additionally, when at restaurants individuals should inquire if sauces or marinades have peanuts since this is a common ingredient in Asian and Mexican food.

Tree Nuts

Tree nuts are one of the common food allergies and one of the food allergies most frequently linked to anaphylaxis (potentially life-threatening reaction that impairs breathing and can send the body into shock). Tree nut allergy triggers are tree nuts (i.e. walnuts) or tree nut products such as nut oil and butters. Tree nut allergies are often confused with peanut allergy; peanuts are legumes, not true tree nuts. Tree nuts are often used as garnishes in a salads, baking mixtures, sauces, breading, desserts, especially in Asian cuisines, and as an ice cream topping. Milk

Milk is one of the eight allergens with specific labeling requirements under the Food Allergen Labeling and Consumer Protection Act of 2004. This act requires manufacturers of packaged food products sold in the United States, which contain milk as an ingredient to include the presence of milk or milk products, in clear language, on the ingredient label.

Lactose

Lactose is a sugar that is found only in milk but can also exist in dairy products and foods that are made with milk derivates. Lactose intolerance affects between 30 million and 50 million Americans. Foods that commonly contain lactose are cakes, biscuits, cheese sauce, cream soups, custard, milk chocolate, pancakes, scrambled eggs, quiche, etc. According to Medical News Today, lactose intolerant Individuals needs to check food carefully, because some foods may contain “hidden lactose.” Such food products include bread, breakfast cereals, margarine, boiled candies, processed meats, and salad dressing. The only treatment for a person with lactose intolerance is to avoid foods that contain lactose.

Egg

According to the American College of Allergy, Asthma, and Immunology, eggs are one of the most common food allergens. Individuals will most likely be allergic to all types of eggs, such as chicken, goose, duck, turkey, or quail. The only way to manage an egg allergy is to avoid eating all types of eggs. Eggs are a hidden ingredient in many foods, including canned soups, salad dressing, ice cream, and meat-based dishes such as meatballs and meatloaf. This means individuals must ensure they are reading all ingredients of the foods they are purchasing.

Wheat

Wheat allergies develop when the body's immune system becomes sensitized and overreacts to something in the environment. In this case, the offending agent is wheat, and it is not life threatening to most people. However, for individuals with Celiac disease, consuming wheat can damage the small intestine. The wheat grain is typically found in countless foods such as cereals, pastas, bread, crackers, hot dogs, beers, sauces, and ice creams or nonfood items with wheat-based ingredient such as Play-Doh, cosmetics, or bath products. Management of wheat allergy involves avoiding food and other products that contain wheat and can trigger symptoms.

Soy

Soy is among the most common food allergens and can be dangerous. Like peanuts, epinephrine is the first-line treatment for anaphylaxis. Soy or derivatives of soy are found in some infant formulas, canned broths, canned tuna, processed meats and hot dogs, energy bars, baked goods and many other processed foods. Additionally, soy is a common ingredient in Asian cuisine and is sometimes contained in chicken nuggets, low-fat peanut butter, alternative nut butter, and even vodka.

Fish

In most cases, fish allergies do not become apparent until adulthood, according to the American College of Allergy, Asthmas, and Immunology. Having a fish allergy does not mean that you are allergic to shellfish, which is a distinctive type of allergy. Fish is one of eight allergens with specific labeling requirements under the Food Allergen Labeling and Consumer Protection Act of 2004. Packaged food products sold in the United States that contain fish or fish products as an ingredient must identify on the ingredient label, in clear language, the specific type of fish used. As with most food allergies, there is no cure; there is only management, which is to avoid fish and fish products by reading food labels carefully. For example, many consumers are unaware, that numerous food items that contain some form of fish. According to the NL Allergy and Immunology Specialist Physicians, some foods that contain or may contain fish:
Barbecue sauce
Gelatin
Sauce
Caesar salad dressing
Hot dogs
Soups
Deli meat
Imitation crab/lobster
Spring rolls
Dips
Marshmallows
Sushi
Fish mixtures
Paella
Tarama
Fried rice

Pizza toppings

Worcestershire sauce

Garnishes

Salad dressing

Wine/Beer fining agent

Shellfish (Shrimps, crabs, lobster, clams, mussels, oysters, scallops)

Shellfish are among the most common allergens in the United States. Fortunately, shellfish is an ingredient that is rarely “hidden” in foods. Shellfish may be found in fish stock, seafood flavoring (for example, crab extract), sushi and surimi. Crustacean shellfish are one of the eight allergens that fall under the labeling requirements of the Food Allergen Labeling and Consumer Protection Act of 2004. This means manufacturers of packaged food items sold in the United States and containing crustacean shellfish, or a crustacean shellfish-based ingredient must state, in clear language, the presence of crustacean shellfish in the food product. These regulations apply only to crustacean shellfish, such as shrimp, lobster and crab, and exclude mollusks, such as oysters, scallops and clams.

Oats

It is not common to have an oat allergy, but it can occur. Common foods and products to avoid because they contain oats are oat flour, oat milk, granola bars, cereal, pudding, beer, and cookies. Oats can be hidden in other foods, so it is extremely important to read ingredients labels thoroughly. This allergy can mimic influenza-like symptoms: dry cough, sneezing and nasal congestion or they can cause abdominal cramping and diarrhea that mimic gluten sensitivity.

Diagram A: Individuals in the United
States that have a specific allergy

| Type of Allergy | Population | |
|---|---|---|
| Lactose | 30 to 50 | million |
| Shellfish | 8.2 | million |
| Milk | 6.1 | million |
| Peanut | 6.1 | million |
| Tree Nut | 3.9 | million |
| Egg | 2.6 | million |
| Wheat | 2.4 | million |
| Soy | 1.9 | million |

| | |
|---|---|
| Oats | No data exist as this is a rare food allergy |

Food allergies can range from mild to serious based on the person and the food item causing the allergic reaction. There is an important difference between an adverse reaction and an allergy. An adverse reaction is defined as symptoms which are considered to be idiosyncratic reactions (undesirable conditions the body experiences from a food substance e.g. the hot flash from ingesting a chili pepper or the odor produced from ingesting an onion). An allergic reaction is defined as symptoms which consist of wheezing, shortness of breath, cramping abdominal pain, blood pressure changes, and the most severe - anaphylaxis. These are all in response to the immune system's reacting to a normally harmless substance e.g. milk, egg, wheat, nuts; and can range from mild to life threatening.

Every three minutes, a food allergy reaction sends someone to the emergency room. Childhood hospitalizations for food allergy tripled between the late 1990s and the mid-2000s. More than 40 percent of children with food allergies have experienced a severe allergic reaction such as anaphylaxis. Medical procedures to treat anaphylaxis resulting from food allergy increased by 380 percent between 2007 and 2016.

Reviewing how someone with a food allergy would experience shopping compared to someone without a food allergy can provide a small sample of the impact of living with and without food allergies. Outside of the financial impact, a person who does not have to take food allergies into consideration when shopping for food items can simply pick up an item, review the packaging briefly, and move on to the next item. For someone with a food allergy, there is more time spent with the food product. They must review the label which has been provided (if the item is applicable to have one under FALCPA), understand the label (the item may be listed by common name, ingredient name, or both), then determine based on their allergy history if this item will be permissible to consume. Additionally, even once a person knows which food items are acceptable and/or avoidable, time is still spent reviewing the label in the event the label has been updated. The extra time spent per item for this one person, in addition to shopping for a family with a variety of allergens, can be significant. Food allergy can limit a major life activity and may qualify an individual for protection under the Americans with Disabilities Act of 1990 (ADA) and Section 504 of the Rehabilitation Act of 1973. Caring for children with food allergies costs U.S. families nearly $25 billion annually. About one in three children with a food allergy reports being bullied as a result. For example, one of the co-inventors of the instant application has a rare allergy to oats. This took many months of elimination dieting to diagnose because oats, oat bran, oat fiber, and oat flour are in all types of products that seem completely unrelated, like ice cream, bread, cookies, beer, etc. It is often included as a filler or as a very small ingredient. It becomes burdensome and time-consuming to try any new food item, because he must read all the ingredients, which are typically in small print, in hopes of identifying the product as safe or avoidable due to the oat by-product ingredient listed. This leads to needing multiple breads, cookies, etc. for family vacations and having to be extremely careful of products in restaurants or beer gardens.

One would hope there will be a cure for food allergies, but as of today no cure exists. The only way to deal with food allergy is avoiding foods that can cause allergies and learn how to recognize and treat the reaction.

U.S. Patent Application 2004/0078218 (Badinelli) discloses a system and apparatus for a consumer to determine food/medicine interactions on a real-time basis. U.S. Pat. No. 6,652,455 (Kocher) and U.S. Pat. No. 5,478,989 (Shepley) analyze products for health information. Several Patent Applications, including US 2016/0085923 (Lacombe et al), US 2020/0250783 (Gnakou), JP2005/018181 (Goto et al) have to do with analyzing restaurant menus for food allergy information. Others are meal planning systems: US 2018/0314804 (Gorre et al) and US 2002/0046060 (Hoskyns).

### SUMMARY

A principal object of the present disclosure is to provide an application program that will assist consumers in identification of food items that may contain ingredients to which they are allergic.

Another object of the present disclosure is to provide an application program that will scan a barcode or QR code on a food product and provide notification of the presence or absence of an allergen in the product's ingredients.

Yet another object of the present disclosure is to provide an application program that will scan a barcode or QR code on a food product and consult the Food and Drug Administration's (FDA) database of all food ingredients to provide notification of the presence or absence of an allergen in the product's ingredients.

A further object of the disclosure is to provide an application to provide notification of the presence or absence of an allergen in a product's ingredients, wherever a barcode or QR code can be found on a product.

A further object of the disclosure is to provide an application to provide notification of the presence or absence of an allergen in a grocery store product's ingredients.

A still further object of the disclosure is to provide an application to provide notification of the presence or absence of an allergen in a grocery store product's ingredients and integrate with other health application programs.

In accordance with the objects of the disclosure, a non-tangible computer readable storage medium is described having instructions that when executed by a processor cause the processor to perform the following operations:
accepting entry of one or more allergens by the user,
accepting entry of a product identification for a product wherein the product identification is correlated to a database of product information including product ingredients,
comparing the product ingredients to the one or more allergens entered by the user, and
notifying the user of presence or absence of allergens in the product.

Also in accordance with the objects of the disclosure, a system for determining the presence or absence of allergens in a product is described. A memory is provided having at least one region for storing computer executable program code. A processor is provided for executing the program code stored in the memory, wherein the program code comprises:
accepting entry by the user of one or more allergens,
accepting entry of a product identification for a product,
finding a product matching the product identification in a database of product information including product ingredients,
comparing the product ingredients of the product to the one or more allergens entered by the user, and
notifying the user of presence or absence of any of the one or more allergens in the product.

Also in accordance with the objects of the disclosure, a method for determining the presence or absence of allergens in a product is described. A user enters one or more allergens. If one of the allergens is not on a list of approved allergens, a form is presented to the user to be filled out and submitted for each of the allergens not on the list. The user will be notified later whether or not the allergens were added to the list. The user enters a product identification for a product. A product is found in a database of product information including product ingredients matching the product identification entered. The product ingredients of the product are compared to the one or more allergens entered by the user. The user is notified of presence or absence of their allergens in the product.

### BRIEF DESCRIPTION OF THE DRAWINGS

In the accompanying drawings forming a material part of this description, there is shown:

FIG. 1 is a flowchart for setting up an account in a preferred embodiment of the present disclosure.

FIG. 2 is an example of a home screen in a preferred embodiment of the present disclosure.

FIG. 3 is a flowchart of the scanning process in the preferred embodiment of the present disclosure.

FIGS. 4A and 4B are examples of listing of previously scanned items in the preferred embodiment of the present disclosure.

FIGS. 5A and 5B are examples of searching a listing of previously scanned items in the preferred embodiment of the present disclosure.

FIGS. 6 and 7 are a block diagrams of a first preferred embodiment of the present invention.

FIG. 8 is a block diagram of a second preferred embodiment of the present invention.

### DETAILED DESCRIPTION

The instant disclosure is most likely to be implemented as a software program, or application, that can run on a hardware device such as a smart phone, tablet, smart device, or other handheld electronic device. Applications that are designed to run on mobile devices are commonly referred to as apps, and can be easily downloaded by a user to a mobile/smart device. Throughout this disclosure, use of the disclosure in a software program will be referred to as the application program, and the hardware platform on which it runs as a mobile device.

The present disclosure describes an application program which can assist consumers in identification of food items that may contain ingredients to which they are allergic. The application program can identify both the common/usual name and specific allergen in a food. This could give people who live with allergies greater access to a wider variety of food items since they can easily identify the presence or absence of a particular allergen instead of merely a generalized category of allergens. By focusing on the top allergens as identified by the law, the application program can begin to collate the data for users from national databases to provide the best recommendation possible based on user profile input to include common and/or allergen names to provide the most accurate profile creation for cross referencing.

The market does not seem to provide comprehensive allergy coverage as existing applications search for broad categories that seem to be what will satisfy many users. There is no ability to enter specific food allergies or foods that trigger adverse reactions or to cross-reference specific allergens to ingredient lists. Furthermore, the ability for users to use their own mobile devices to check for allergens in any food item before purchase would be a great advantage.

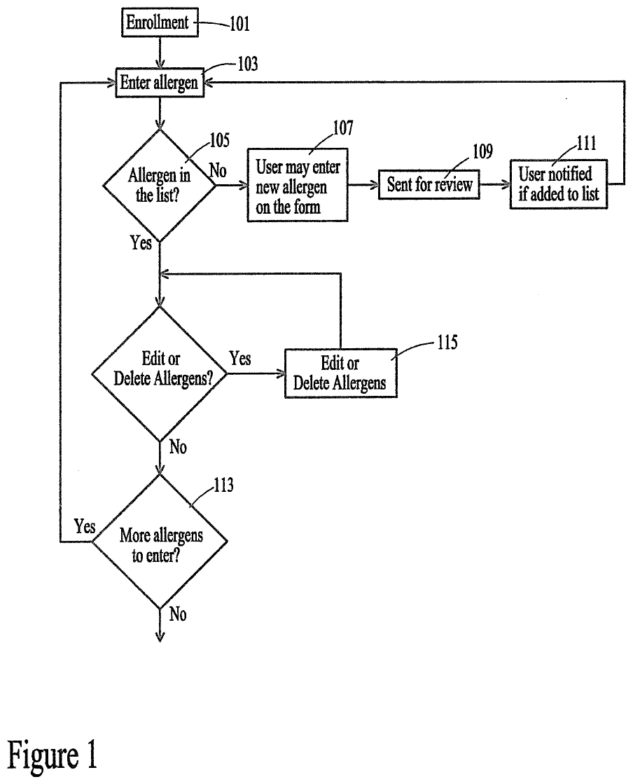

The application program of the present disclosure can be downloaded onto a mobile device. The mobile device could be a smart phone, electronic tablet, pad, or other handheld electronic device. A user will create a personalized account 101 on the application, as illustrated in the flowchart in FIG. 1. Then, into the search feature, the user will enter their specific allergen 103, selecting from a list of allergens. If the user's allergen is not on the list 105, the user will fill out a form 107 to enter their allergen for review 109. An internal peer-review will use nationally accredited sources as guidelines to determine whether or not the allergen will be entered into the application's database. Based on the internal review, the user will be notified 111 whether or not the new food allergen will be entered into the application's database. The notification may be sent to an email address the user supplied during the enrollment step. The user will continue entering allergens 113 until the profile is complete. Allergens may be edited or deleted 115.

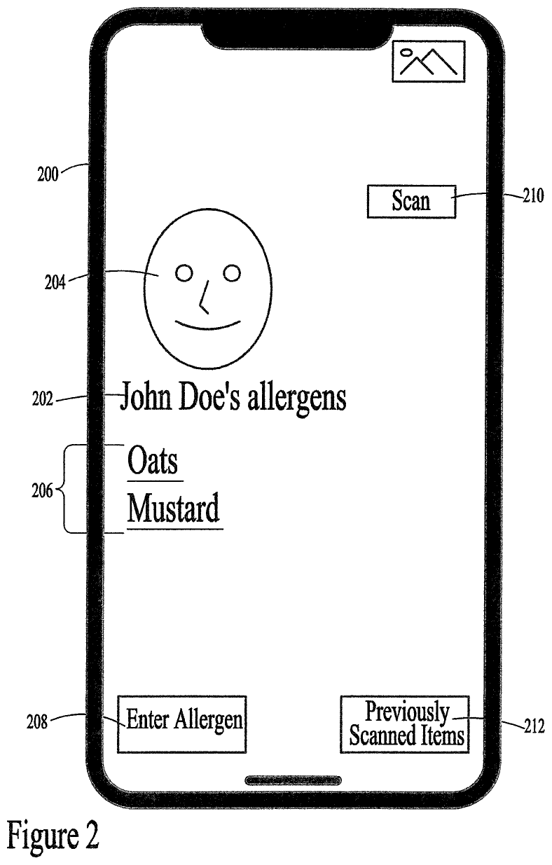

A sample home page 200 of the application for a user is illustrated in FIG. 2. For example, the screen will show the user's name 202, an optional photograph 204, and a list of allergens 206. Options on the screen are to enter a new allergen 208, access the camera on the mobile device to scan a food item 210, and view previously scanned items 212. It will be understood that screen shots shown are samples only and may differ in layout.

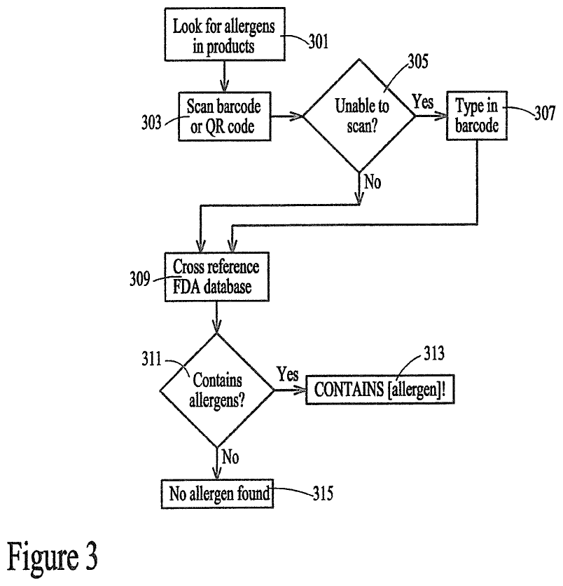

Referring to the flowchart of FIG. 3, to determine if a food item contains one or more of the user's allergens 301, the user will scan a barcode or QR code on the food item 303. If the camera is unable to scan the barcode or QR code 305, the user can manually enter the barcode number 307. The search will cross reference the Food and Drug Administration (FDA) database 309 for that scanned food item. Prior attempts to allow consumers to, for example, determine food/medicine interactions, have failed to provide any technological way for consumers to access information in the FDA database. For example, the food item may be a frozen food, alcohol, pasta, canned good, chocolate, beans, peas, chips, salsa, or any food with a barcode or QR code. The search compares the user's allergen list against all the ingredients listed in the FDA database for that food item 311. If any of the ingredients are flagged as containing the user's allergen, a message 313 will appear on the phone screen, for example, in bold red letters: CONTAINS [ALLERGEN NAME] (e.g. CONTAINS OATS!) along with an in-app notification. If no allergen is identified, the user will receive a message 315 stating, for example, in green lower-case letters: “No Allergen Found” along with an in-app notification. The in-app notification will assist users who require accessibility features, such as with an audio message, for example.

On the U.S. Food and Drug Administration (FDA) website (aaccessdata.fda.gov) the Substances Added to Food inventory includes the following types of ingredients regulated by the U.S. Food and Drug Administration:

Food additives and color additives that are listed in FDA regulations (21 CFR Parts 172, 173 and Parts 73, 74 respectively), and flavoring substances evaluated by FEMA* and JECFA*.

Generally Recognized as Safe (“GRAS”) substances that are listed in FDA regulations (21 CFR Parts 182 and 184).

Substances approved for specific uses in foods prior to September 6, 1958, known as prior-sanctioned substances (21 CFR Part 181).

Substances formerly used:

Prohibited substances that are listed in FDA regulations (21 CFR Part 189) as prohibited from use in food (labeled as “PROHIBITED” or “PROHIBITED WITH EXCEPTIONS”).

Delisted color additives in FDA regulations (21 CFR 81.10 and/or 81.30) (labeled as “DELISTED”).

Some substances “no longer FEMA GRAS”

This database is downloadable to a searchable database in Excel format. The application program backend software will search the FDA database. It is anticipated that updates to the FDA database will be provided to the backend software. It is possible that other food ingredient databases may also be used, for example, from other third parties besides the FDA.

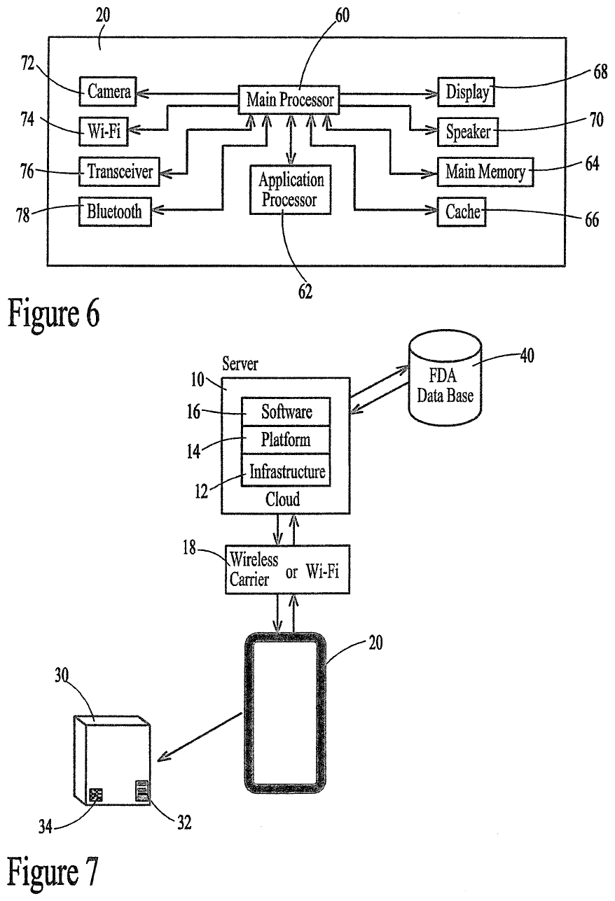

FIG. 6 is a block diagram of a typical mobile device 20 on which the disclosed application program would run. Mobile device 20 could be a smart phone, electronic tablet, pad, or other handheld electronic device. For example, the mobile device 20 may include a main processor 60, an applications processor 62, a main memory 64, a cache memory 66, a display 68, a speaker 70, a camera 72, a Wi-Fi connection 74, a transceiver 76, and a Bluetooth connection 78, for example, all in communication with the main processor 60. The main processor 60 is any logic circuitry that responds to and processes instructions received from the main memory 64. The main memory 64 may include one or more memory chips capable of storing data and allowing any storage location to be directly accessed by the main processor 60.

FIG. 7 illustrates a cloud-based computing environment where mobile device 20 is in communication with cloud 10 (typically implemented as one or more servers) over one or more networks. The cloud 10 may be a public, private, or hybrid cloud. The cloud may include infrastructure 12 providing networks, servers, and storage, a platform 14 servicing the functionality of the infrastructure, and software 16 implementing the services. The servers may be resident and/or executed on any type of computing device that can communicate on any type of network and can perform the operations herein described. The application program may be used with any publishing platform. The application program has both a client portion, running on mobile device 20, and a server portion, as represented by application program 16 running on cloud 10. Mobile device 20 is in communication with the server-side application program 16 through wireless carrier or Wi-Fi 18. Product 30 having UPC code 32 and/or QR code 34 is scanned by the camera in the mobile device 20. The FDA database 40 is accessed by the application program software 16.

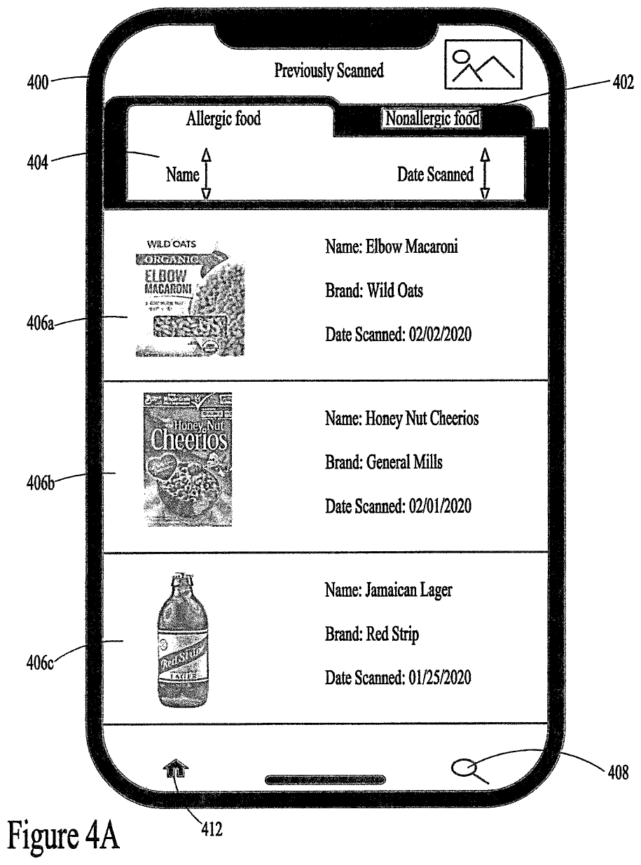

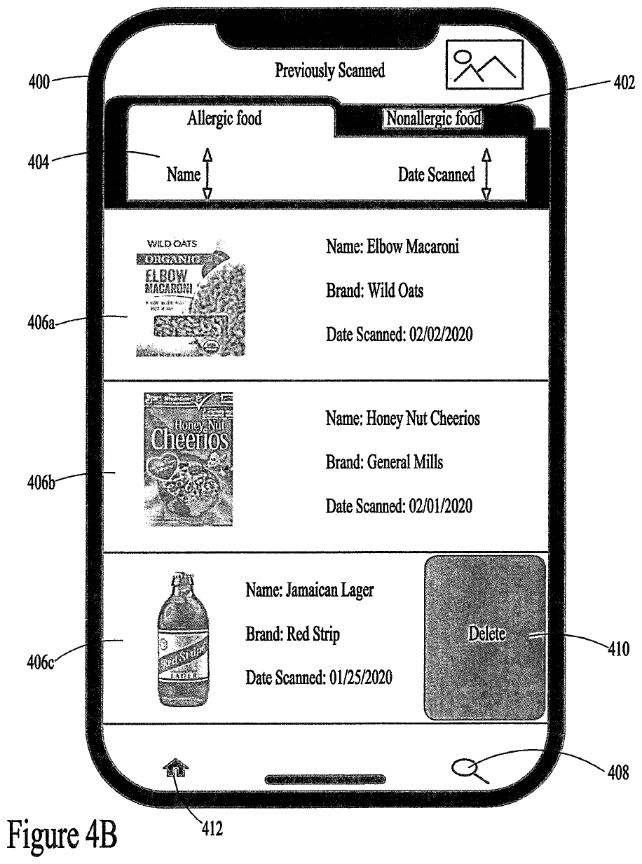

The user will be able to access the food item(s) previously scanned. An example of the previously scanned item display page 400 in shown in FIGS. 4A and 4B. The previously scanned items page will be divided into two tabs: nonallergic food 402 and allergic food 404, defaulting to show the nonallergic food tab, for example. FIG. 4A shows the allergic food tab 404. The food items (406a, 406b, 406c) will be displayed with the Name; Brand; and Date Scanned. The user will have the option to sort alphabetically or by date scanned on both tabs. The default display will be by date scanned. Pressing the home icon 412 returns the user to the home screen as shown in FIG. 2.

The user can delete a previously scanned food item, for example, by swiping right next to an item to delete it from the list 410, as shown in FIG. 4B. This function can also be used to delete a food item during the Search function. On the Previously Scanned pages, a user can search for any of the previously scanned food items. Using the search feature 408, the user can search for the food item by name and/or brand name.

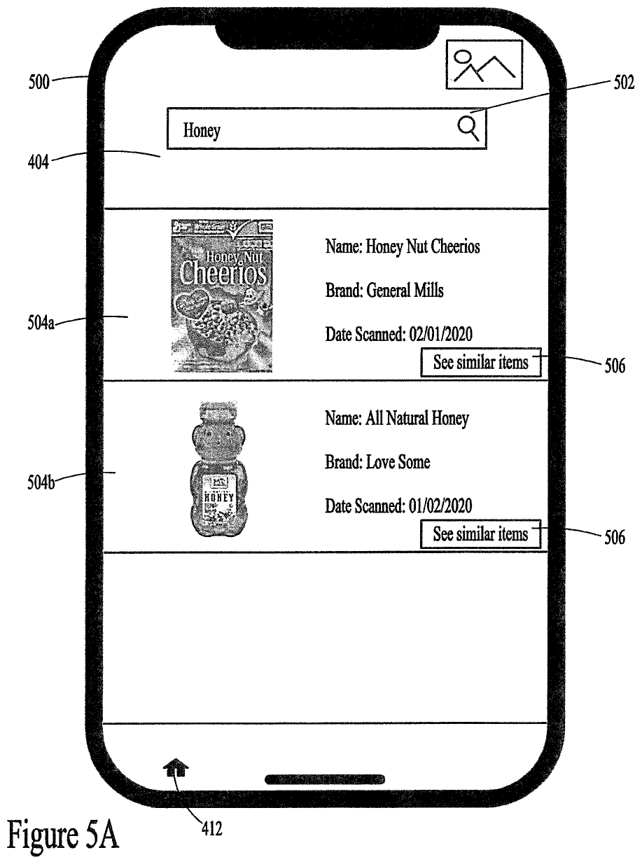

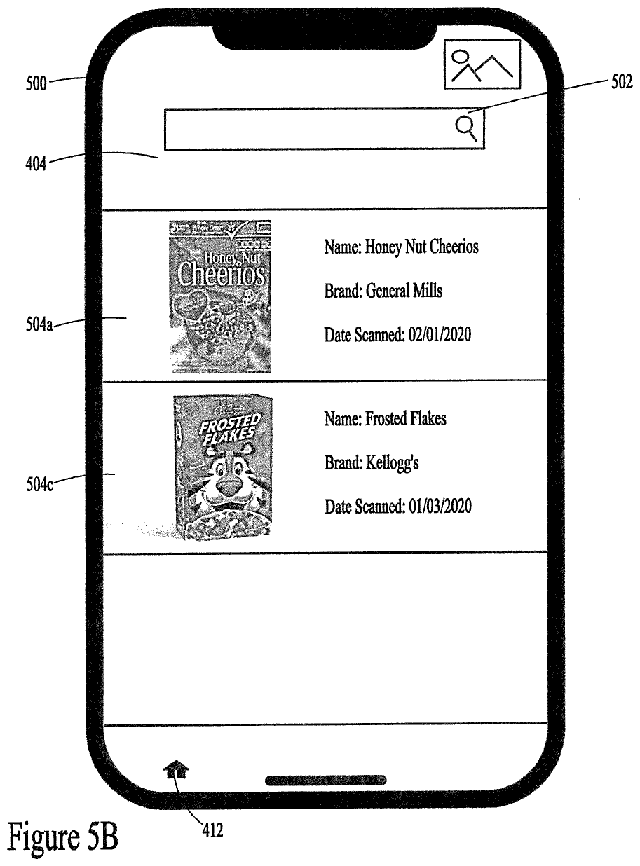

FIG. 5A illustrates a sample search screen 500. The user can search for a food by name, for example “honey” as shown in the search box 502. Food items previously scanned that contain the word “honey” in their names are shown 504a, 504b. The user can click on the “see similar items” button 506 for one of the items to see additional food items in that same category. For example, if the user clicks the “see similar items” button for Honey Nut Cheerios (trademark of General Mills IP Holdings I, LLC), as shown in FIG. 5B, the screen will show all scanned cereals 504a, 504c, for example, that contain any of the user's allergens.

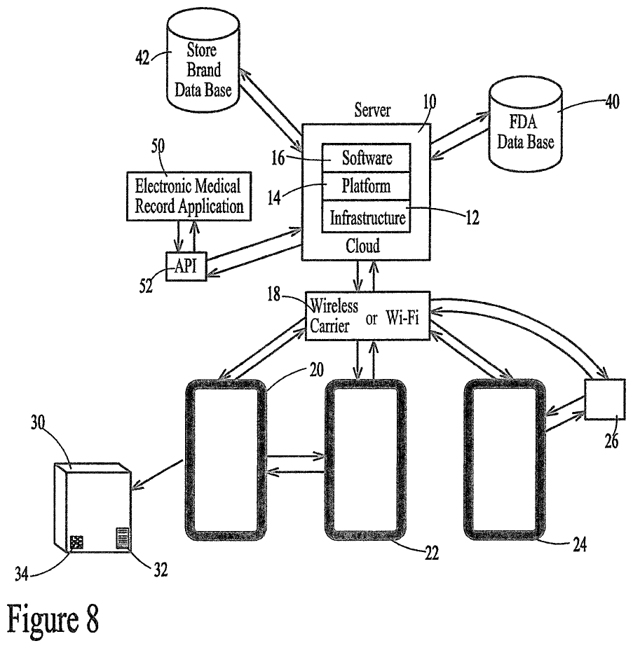

FIG. 8 illustrates a block diagram with additional features. Preferably, the application program will be integrated with grocery stores that have their own brands. For example, a store brand data base 42 might be accessed by the backend application software in addition to the FDA database. It would be desirable also to integrate the application program with other Health application programs. For example, medical record application programs could include a link to access the application program to provide the user's allergy profile as part of the medical records. Weight or food applications could link to the application program to look for foods to avoid based on the user's allergy profile. It would be desirable to link the application program to mobile device hands free applications 26 such as Siri (registered trademark of Apple Corporation) so that these applications can access information in the application program and launch the application if requested.

Additional features include the ability to enter a user's allergic reaction such as rash, hives, diarrhea, vomiting, etc. to each allergen. The user's allergic reaction can be transferred to a patient's personal health record where the user's healthcare team can have access to the information. If a user would like to share their list of allergens with a family member such as a spouse, partner, parent, etc. they can ‘share’ via text message, email, airdrop, etc. A link will be sent to the family member's smart phone 22 with the ability to “read only” the allergy information. The application program will have the capability to share the user's allergy(ies) with an Electronic Medical Record system 50 through the use of an application programming interface (API) 52. The user will be able to send their food allergens to their record that is used by a physician and/or hospital. If a parent/caregiver has a child under the age of 18, the parent/caregiver can create a child account under their main the application program account. This will give the parent/caregiver the ability to identify food item(s) that their child may be allergic to. If the user notices a flare up based on what he/she eats (e.g. psoriasis correlated to night shade), the user has the ability to share this information with their healthcare providers. Additionally, the types of food the user scanned could be shared with Nutrition Counselors.

Furthermore, the application program will have the capability to require authorization to access the application such as by facial recognition on a mobile device or by requiring the entry of a pin code. Alternatively, authorization may be via an email program or other method.

The application program of the present disclosure will enable a shopper to scan a barcode or QR code on a food product and connect to the Food and Drug Administration's (FDA) database of all food ingredients to provide notification of the presence or absence of an allergen in the product's ingredients. Reviewing foods with this application could give users time back by streamlining their experience while they shop. Additionally, the application program can be used to provide notification of the presence or absence of an allergen in a product's ingredients, wherever a barcode or QR code can be found on a product. That is, anywhere products are sold, such as convenience stores, or anywhere packaged products are found, such as in a user's pantry or in a friend's pantry.

While the disclosed subject matter has been described in conjunction with a number of embodiments, it is evident that many alternatives, modifications and variations would be, or are, apparent to those of ordinary skill in the applicable arts. Accordingly, Applicant intends to embrace all such alternatives, modifications, equivalents and variations that are within the spirit and scope of the disclosed subject matter and the appended claims.

1. A non-tangible computer readable storage medium having instructions that when executed by a processor cause said processor to perform operations comprising:
accepting entry of one or more allergens by a user;
accepting entry from a mobile device of a product identification for a product wherein said product identification is a barcode or QR code scanned into said mobile device by a camera that is part of said mobile device and wherein said barcode or QR code is used to find said product in to a food ingredient database including all product ingredients;
comparing said product ingredients to said one or more allergens entered by said user; and
notifying said user of presence or absence of allergens in said product.

2. The non-tangible computer readable storage medium according to claim 1 further comprising setting up a user profile for said user wherein said one or more allergens entered by said user are stored in said user profile.

3. The non-tangible computer readable storage medium according to claim 1 wherein if any of said one or more of allergens is not on a list of approved allergens, then presenting a form to said user to be filled out and submitted for each of said allergens not on said list, wherein said user will be notified later whether or not said allergens were added to said list.

4. (canceled)

5. The non-tangible computer readable storage medium according to claim 1 wherein said food ingredient database is a Food and Drug Administration food ingredient database.

6. The non-tangible computer readable storage medium according to claim 1 wherein said notifying said user is by one or more of: a printed message on a smart device and an in-app notification.

7. The non-tangible computer readable storage medium according to claim 1 further comprising:
displaying products for which product identification entries have been accepted;
maintaining a first list of said products containing at least one of said allergens;
maintaining a second list of said products containing none of said allergens; and
accepting input of a search term to search in either of said first or second lists for said search term and displaying said products having said search term in their names

8. The non-tangible computer readable storage medium according to claim 2 further comprising requiring authorization to access said user profile.

9. The non-tangible computer readable storage medium according to claim 1 further comprising allowing sharing of said one or more allergens by said user with other users or with other application programs.

10. The non-tangible computer readable storage medium according to claim 1 wherein said food ingredient database is one or more of: a Food and Drug Administration food ingredient database or any of a plurality of store brand food ingredient databases or other third-party food ingredients databases.

11. The non-tangible computer readable storage medium according to claim 1 further comprising entering allergic reactions to any of said one or more allergens.

12. A system for determining the presence or absence of allergens in a product comprising:
a memory having at least one region for storing computer executable program code; and
a processor for executing the program code stored in said memory, wherein said program code comprises:
accepting entry by a user of one or more allergens;
accepting entry from a mobile device of a product identification for a product wherein said product identification is a barcode or QR code scanned into said mobile device by a camera that is part of said mobile device;
finding a product matching said product identification in a food ingredient database of including all product ingredients;
comparing said product ingredients of said product to said one or more allergens entered by said user; and
notifying said user of presence or absence of any of said one or more allergens in said product.

13. The system according to claim 12 further comprising setting up a user profile for said user wherein said one or more allergens entered by said user are stored in said user profile.

14. The system according to claim 12 wherein if any of said one or more allergens is not on a list of approved allergens, then presenting a form to said user to be filled out and submitted for each of said allergens not on said list, wherein said user will be notified later whether or not said allergens were added to said list.

15. The system according to claim 12 wherein said user enters said one or more allergens on a mobile device.

16. (canceled)

17. The system according to claim 16 wherein said notifying said user is by one or more of:
displaying a printed message on said smart device; and
providing an in-app notification on said smart device.

18. The system according to claim 12 wherein said food ingredient database is one or more of: a Food and Drug Administration food ingredient database or any of a plurality of store brand food ingredient databases or other third-party food ingredient databases.

19. The system according to claim 15 further comprising:
on said mobile device,
displaying products for which product identification entries have been accepted;
maintaining a first list of said products containing at least one of said one or more allergens;
maintaining a second list of said products containing none of said one or more allergens;
accepting input of a search term to search in either of said first or second lists for said search term and displaying said products having said search term in their names; and
providing the ability to display products in either said first or second lists that are in a same category as a chosen product.

20. A method for determining the presence or absence of allergens in a product comprising:
accepting entry by a user of one or more allergens wherein if one of said one or more allergens is not on a list of approved allergens, then presenting a form to said user to be filled out and submitted for each of said allergens not on said list, wherein said user will be notified later whether or not said allergens were added to said list;
accepting entry from a mobile device of a product identification for a product wherein said product identification is a barcode or QR code scanned into said mobile device by a camera that is part of said mobile device;
finding a product matching said product identification in a food ingredient database including all product ingredients;
comparing said product ingredients of said product to said one or more allergens entered by said user; and
notifying said user of presence or absence of any of said allergens in said product.

21. The method according to claim 20 further comprising setting up a user profile for said user wherein said one or more allergens entered by said user are stored in said user profile.

22. (canceled)

23. The method according to claim 20 wherein said food ingredient database is one or more of: a Food and Drug Administration food ingredient database or any of a plurality of store brand food ingredient databases or other third-party food ingredient databases.

24. The method according to claim 20 wherein said notifying said user is by one or more of: a printed message on a mobile device and an in-app notification.

25. The method according to claim 20 further comprising:
displaying products for which product identification entries have been accepted;
maintaining a first list of said products containing at least one of said allergens;
maintaining a second list of said products containing none of said allergens; and
accepting input of a search term to search in either of said first or second lists for said search term and displaying said products having said search term in their names.
